(19) United States
(12) Patent Application Publication
(10) Pub. No.: US 2005/0261592 A1
(43) Pub. Date: Nov. 24, 2005

(54) Fluorescence endoscope apparatus

(76) Inventor: Takeshi Suga, Tokyo-to (JP)

Correspondence Address: KENYON & KENYON, 1500 K STREET NW, SUITE 700, WASHINGTON, DC, 20005, US

(21) Appl. No.: 11/129,472

(22) Filed: May 16, 2005

(30) Foreign Application Priority Data
May 21, 2004 (JP) 2004-151646

(51) Int. Cl. A61B 6/00

(52) U.S. Cl. 600/478; 600/473

(57) ABSTRACT

A fluorescence endoscope apparatus has a light source apparatus having at least a light source, an optical filter unit for exciting light, and a plurality of optical filter units for normal illumination light, an electronic endoscope which picks up an image of fluorescence and a plurality of reflecting light obtained from the object, and an image processing apparatus which processes an image signal of a fluorescence image and a plurality of reflecting light images picked up by the electronic endoscope, and delivers them to a monitor. The image processing has a first color control means which carries out a color control of only the image signals of a plurality of the reflecting light images based on an image signal obtained by using a standard object as an object, and a second color control means which carries out a color control of an image signal of said plurality of reflecting light images adjusted by the first color control means and an image signal of the fluorescence image obtained by using a living tissue as an object on the basis of the image signal obtained by said image processing apparatus using the living tissue as an object.

This application claims benefits of Japanese Application No. 2004-151646 filed in Japan on May 21, 2004, the contents of which are incorporated by this reference.

### BACKGROUND OF THE INVENTION

1. Field of the Invention

The present invention relates to a fluorescence endoscope apparatus for obtaining a fluorescence image.

2. Description of the Related Art

Conventionally, in medical application field, for example, a fluorescence endoscope apparatus which is composed so that a fluorescence image is obtained in order to identify a normal tissue and a diseased tissue in addition to obtaining an ordinary image by an ordinal white light, has been known.

Such fluorescence endoscope apparatus has been proposed in publications of Japanese unexamined patent application Toku Kai 2001-137174 and Toku Kai 2003-111716.

The fluorescence endoscope apparatus disclosed by Toku Kai 2001-137174 is composed so that an image signal may be generated by reflecting the relative intensity of fluorescence to color, and the intensity of a reference light to brightness.

The fluorescence endoscope apparatus disclosed by Toku Kai 2004-24611 is composed so that the intensity ratio of a fluorescence image signal and a plurality of reflecting light signals may be adjusted by setting zone of a diseased portion and a normal portion of a living body tissue from observation images.

The fluorescence endoscope apparatus disclosed by Toku Kai 2003-2003 is composed so that color control of the fluorescence and reflecting light may be carried out by irradiating a standard light source containing wavelength band of the fluorescence to an inspecting portion of the organism.

### SUMARRY OF THE INVENTION

The fluorescence endoscope apparatus according to the present invention comprises a light source apparatus having at least a light source, an optical filter unit for exciting light, and a plurality of optical filter units for normal illumination light, an electronic endoscope which leads exciting light and a plurality of normal illumination light from the light source apparatus to an object, and picks up an image of fluorescence and a plurality of reflecting light obtained from the object, and an image processing apparatus which processes an image signal of a fluorescence image and a plurality of reflecting light images picked up by the electronic endoscope, and delivers them to a monitor, and the image processing further comprising a first color control means which carries out a color control of only the image signals of a plurality of the reflecting light images based on an image signal obtained by using a standard object as an object.

In the fluorescence endoscope apparatus according to the present invention, the image processing apparatus comprises a second color control means, which carries out a color control of an image signal of said plurality of reflecting light images adjusted by the first color control means and an image signal of the fluorescence image obtained by using a living tissue as an object on the basis of the image signal obtained by said image processing apparatus using the living tissue as the object.

In the fluorescence endoscope apparatus according to the present invention, each of optical filter units for normal illumination light is composed of two sheets of the optical filter which are pasted togerther.

In the fluorescence endoscope apparatus according to the present invention, each of transmittances of the plurality of optical filter units for the normal illumination light is 1/100 or less of the transmittance of the optical filter unit for the exciting light.

According to the present invention, a fluorescence endoscope apparatus for obtaining an image, by which a normal tissue or a diseased tissue is easily identified with simple constitution can be achieved.

These and other features and advantages will become apparent from the following detailed description of the preferred embodiments when taken in conjunction with the accompanying drawings.

### BRIEF DESCRIPTION OF THE DRAWINGS

FIG. 1 is a block diagram showing the whole composition of the fluorescence-endoscope-apparatus concerning the first embodiment of the present invention.

FIG. 2 is a diagram showing a composition of a switching filter unit in which a filter unit for normal observation and a filter unit for fluorescence observation are arranged.

FIG. 3A is a graph showing a transmittance characteristic to wavelength of the filter unit for normal observation.

FIG. 3B is a graph showing a transmittance characteristic to wavelength of the filter unit for fluorescence observation.

FIG. 3C is a graph showing a transmittance characteristic to wavelength of the filter unit for cutting exciting light.

FIG. 4A is a graph showing a characteristic of an intensity of light to wavelength, where the light is received by CCD when a white standard object is observed at a normal observation mode.

FIG. 4B is a graph showing a characteristic of an intensity of light to wavelength, where the light is received by CCD when a skin tissue is observed at a fluorescence observation mode.

FIG. 5A is a graph showing an example of an intensity distribution characteristic obtained from the wavelength of the fluorescence image to a living tissue.

FIG. 5B is a graph showing an example of an intensity distribution characteristic obtained from the wavelength of a reflecting light image to a living tissue.

FIG. 6 is a block diagram showing a composition of an image processing circuit equipped in the fluorescence endoscope apparatus of FIG. 1.

FIG. 7 is a block diagram showing a composition of a setting switch connected to the image processing circuit shown in FIG. 6.

FIG. 8 is a diagram showing an example of an image display when an area of interest is set up to the composite image displayed on the monitor.

FIG. 9 is a diagram showing a modification of a color control switch with which the setting out switch shown in FIG. 7 is equipped.

FIG. 10 is an outline composition diagram of G1 filter unit used for the fluorescence endoscope apparatus concerning the second embodiment of the present invention.

FIG. 11 is a graph showing a transmittance characteristic of the optical filter unit with which G1 filter unit 22b shown in FIG. 10 is equipped.

FIG. 12 is a graph showing a transmittance characteristic of a modification of an optical filter unit shown in FIG. 11.

### DETAILED DESCRIPTION OF THE PREFERRED EMBODIMENTS

Prior to explaining embodiments, function and advantages of the present invention will be explained

An intensity of reflecting light from a patient has a characteristic such that the intensity changes little for every patient but changes depending upon the kind or state of a living tissue.

However, if the first color control means is provided like the fluorescence endoscope apparatus of the present invention, the intensity ratio of only the image signal of the reflecting light image of each wavelength band can be adjusted by using a standardobject of which the state is unchanged. Since the whole state of the standard object is fixed uniformly, it is not necessary to set up a zone in an observation image, and a color control (improving color reproduction) of exact reflecting light is simply carried out. By adjusting the intensity ratio of the intensity of the image signal of this adjusted reflecting light image and the image signal of a fluorescence image, useful diagnostic information can be obtained.

In the present invention, a standard object means a reflective component, for example, a reflecting component, such as a white board or the like, in which the whole state is constantly fixed and a reflection factor characteristic is composed to be uniform within the scope of an observation object.

If a second color control means is provided, like the fluorescence endoscope apparatus of the present invention, in a patient's body (living tissue), the intensity of the image signal of a fluorescence image can be adjusted so that these intensity ratios of the image signal of the fluorescence image obtained by radiating the exciting light for exciting fluorescence and the image signal of the reflecting light image of a plurality of wavelength bands with which the intensity ratio has been adjusted through the first color control means using the standard object may become a predetermined intensity ratio, and accordingly even if a fluorescence intensity differs for every patient, a constant and exact color reproduction becomes possible and a useful diagnostic information can be obtained. Furthermore, if it is composed such that only the intensity of the image signal of the fluorescence image obtained from the living tissue may be adjusted, it is not necessary to set the area of a pathologically changed portion where the setup is difficult, and it is sufficient to carry out a setting of part of only the normal portion of the living tissue which can easily be set, and the color control becomes possible from a value of the image signal of the fluorescence image at the normal portion. Therefore, the color control can be carried out simply and exactly.

As for adjustment of the intensity of the image signal of the fluorescence image obtained from the living tissue in the fluorescence endoscope apparatus of the present invention, it is desirable to adjust so that the intensity of the image signal of the reflecting light image from a standard object, and the intensity of the image signal of a fluorescence image may become a predetermined intensity ratio defined beforehand. Otherwise, it is good to be composed so as to enable to adjust arbitrarily the intensity of the image signal of the fluorescence image to such intensity ratio as a user wants.

Fluorescence obtained in a fluorescence observation is extremely weaker compared with the reflecting light of a normal illumination light. Therefore, in the fluorescence endoscope apparatus of the present invention, it is desirable that transmittances of a plurality of optical filter units for the normal illumination light are set to 1/100 of the transmittance of an optical filter unit for the exciting light or less, the intensity of the normal illumination light for obtaining reflecting light is made small about 1/100 compared with the intensity of the exciting light. By this, the reflecting light intensity of the normal illumination light which reaches CCD and the fluorescence intensity by the exciting light can be made near. Therefore, it is possible to avoid that only the CCD output of the reflecting light is saturated.

If the intensity of the normal illumination light for obtaining reflecting light is made small as mentioned above, the influence by the variation of the transmittance of each optical filter unit used for obtaining the reflecting light of a desired wavelength band becomes large. When the variation of the intensity of the image signal of the reflecting light image in each wavelength band produced by this variation is adjusted electrically by a color control, an electric noise increases and accuracy of the image signal of a reflecting light image deteriorates by such adjustment.

Therefore, in the fluorescence endoscope apparatus of the present invention, it is desirable to compose such that each of the optical filter unit for normal illumination light is formed by sticking two sheets of the optical filter. Moreover, it is desirable that the optical filter unit is composed of a multilayered film coat of dielectric material. By this way, it becomes possible to reduce the variation of the transmittance of a filter unit.

### First Embodiment

Hereafter, embodiments of the present invention will be explained using drawings.

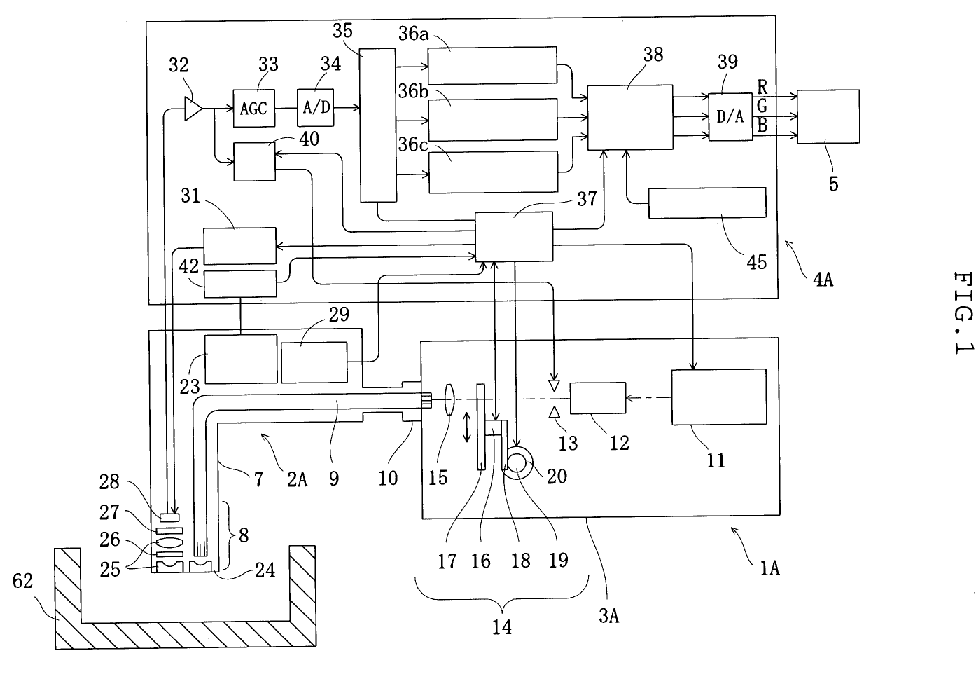

FIG. 1 is a block diagram showing the whole composition of the fluorescence endoscope apparatus concerning the first embodiment of the present invention.

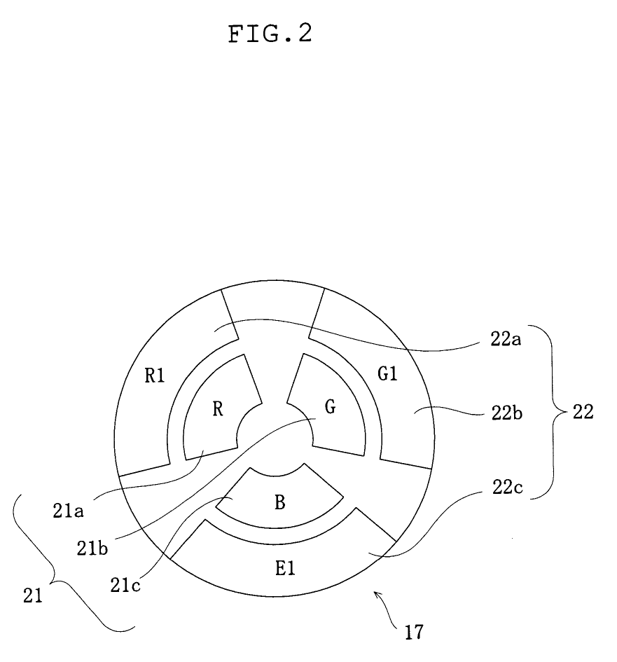

FIG. 2 is a diagram showing a composition of a switching filter unit in which a filter unit for normal observation and a filter unit for fluorescence observation are arranged.

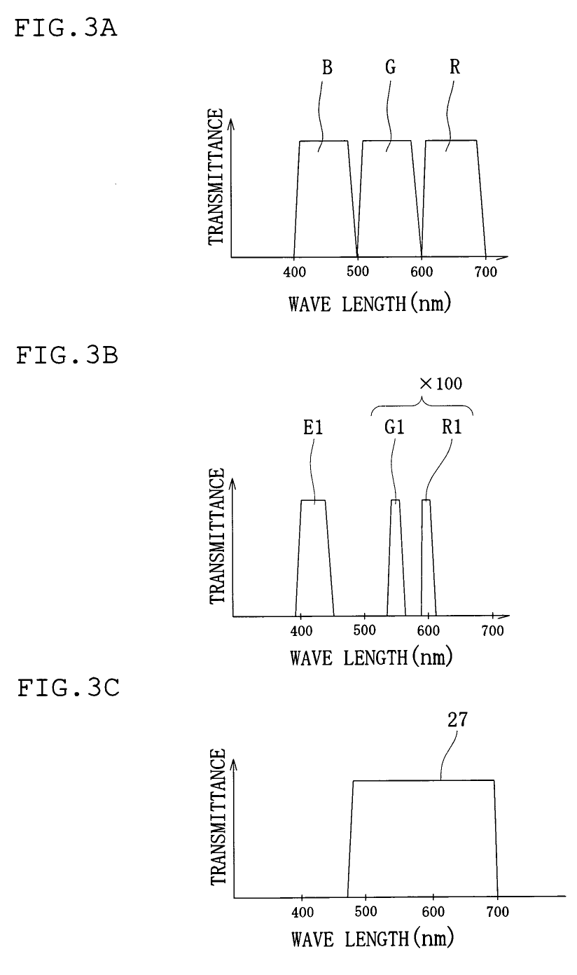

FIG. 3A is a graph showing a transmittance characteristic to the wavelength of a filter unit for a normal observation,

FIG. 3B is a graph showing a transmittance characteristic to the wavelength of a filter unit for a fluorescence observation,

FIG. 3C is a graph showing a transmittance characteristic to wavelength of the exciting light cutoff filter unit.

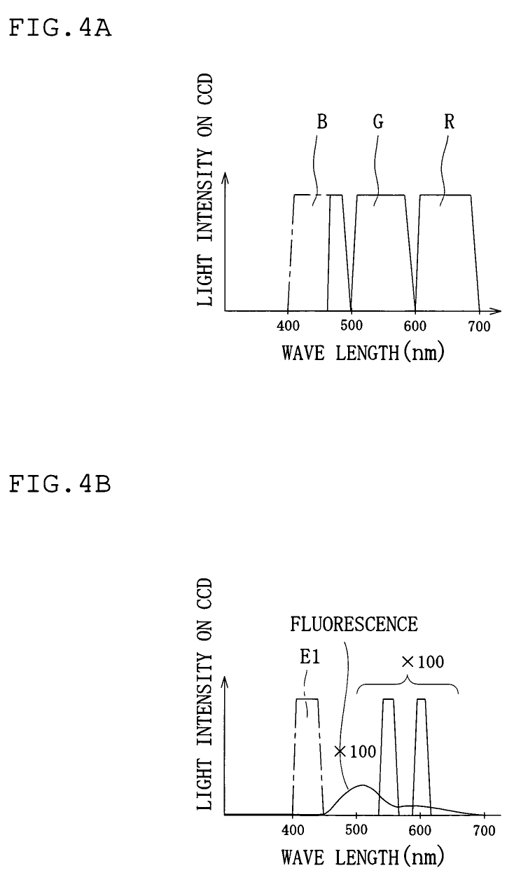

FIG. 4A is a graph showing a characteristic to the wavelength of an intensity of light received by CCD, when a white standard object is observed in normal observation mode.

FIG. 4B is a graph showing a characteristic to the wavelength of the intensity of light received by CCD when a skin is observed by a fluorescence observation mode.

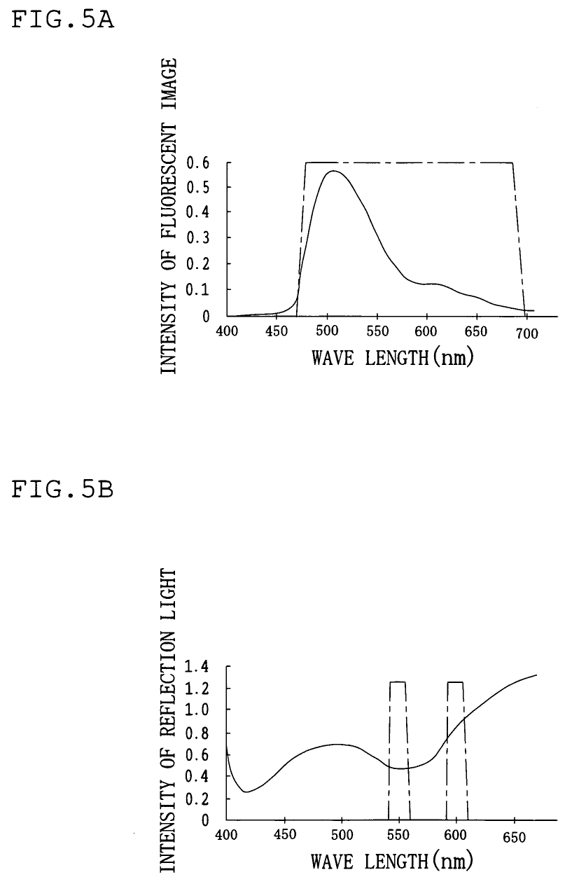

FIG. 5A is a graph showing an example of the intensity distribution characteristic obtained from the wavelength of the fluorescence image to a living tissue.

FIG. 5B is a graph showing an example of an intensity distribution characteristic obtained from the wavelength of a reflecting light image to a living tissue.

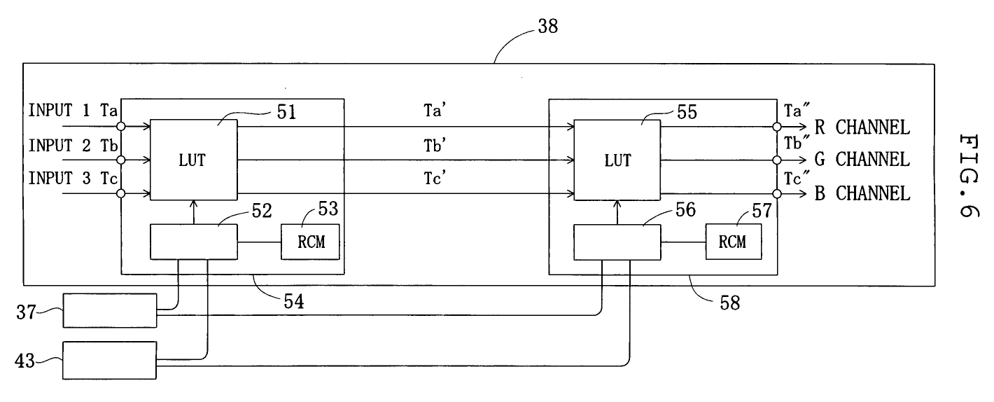

FIG. 6 is a block diagram showing a composition of an image processing circuit equipped in the fluorescence endoscope apparatus of FIG. 1.

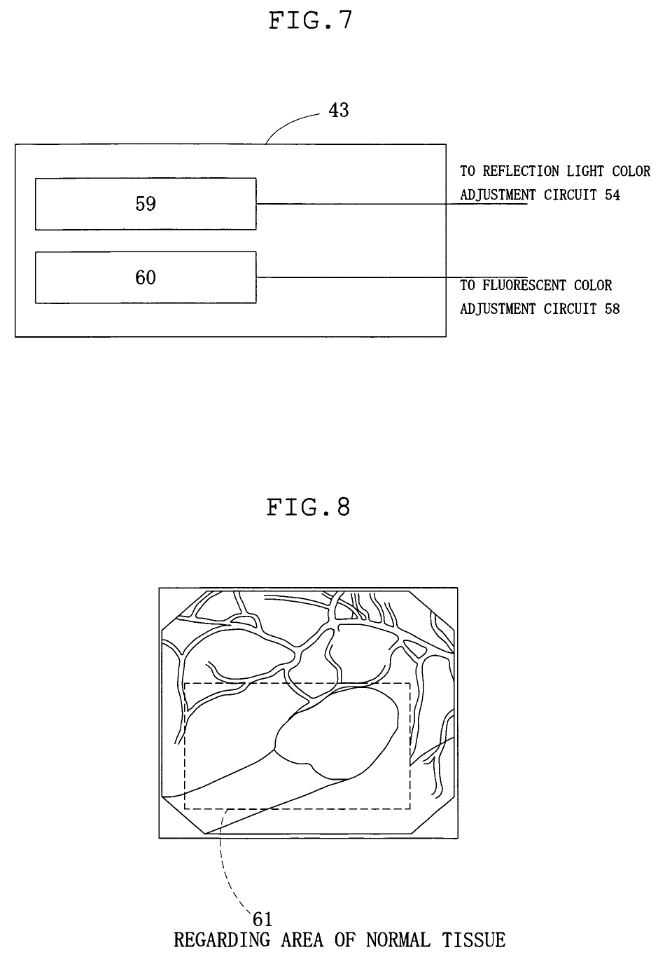

FIG. 7 is a block diagram showing a composition of a setting switch connected to an image processing circuit shown in FIG. 6.

FIG. 8 is a diagram showing an example of an image display when an area of interest is set up to the composite image displayed on the monitor.

A fluorescence endoscope apparatus 1A of the first embodiment comprises an illumination light for a normal observation, a light source apparatus 3A which can selectively emit illumination light for a fluorescence observation, an electronic endoscope 2A which transmits the light from the light source apparatus 3A into an abdominal cavity that is an object, and picks up an image of the fluorescence which is obtained from the object and images of a plurality of reflecting light, an image processing apparatus 4A which carries out signal processing about the image signal from the electronic endoscope 2A, and transmits it to the monitor, the monitor 5 which is able to display the image signal for which a signal processing has been carried out by the image processing apparatus 4A.

An electronic endoscope 2A has an elongated insertion portion 7 inserted into the abdominal cavity that is the object. The insertion portion 7 contains an illumination means and an image pick-up means in a tip end portion 8. Moreover, a light guide fiber 9 which transmits the illumination light for normal observation and the illumination light for fluorescence observations is inserted into the insertion portion 7. The light guide fiber 9 is connected to the light source apparatus 3A, and it is attachably and detachablly connected by a connector 10 for the light source arranged at an light entrance edge located near at hand.

The light source apparatus 3A which is driven so that light may be emitted by a lamp drive circuit 11, comprises a lamp 12 for emitting the light which includes a radiation band from an infrared wavelength band to a visible radiation band, an aperture stop of the light source 13 which is arranged on an illumination light path with a lamp 12, and limits the quantity of the light from the lamp 12, a filter unit switching portion 14 arranged on the illumination light path, a condensing lens 15 for condensing the light which passed along the filter unit switching portion 14.

A filter unit switching portion 14 comprises a switching filter unit 17 which is rotated through a motor 16 for rotation and switches an optical filter unit arranged on a light path through a motor 20 for movement, the motor 20 for movement for moving a switching filter unit 17 in the direction perpendicular to an optical axis with the motor 16 for rotation by rotating a pinion 19 connected by a screw on a rack 18 attached in the motor 16 for rotation.

A switching filter unit 17 as shown in FIG. 2, is composed of a filter unit 21 for a normal observation and a filter unit 22 for a fluorescence observation, each of which is arranged at the inner side of circumference and the outer side of circumference on a concentric circle, respectively. The switching filter unit 17 is composed so as to enable to switch, by driving the motor 20 for movement, a setup of an operating state of the normal image mode (it is also usually called a normal mode), where the filter unit 21 for normal observation is arranged on the light path, a setup of another operating state of the fluorescence image mode (it is also called a fluorescence mode), where an optical filter unit arranged on the light path is switched from the optical filter unit 21 for a normal illumination light to the filter unit 22 for fluorescence observation.

The normal observation filter unit 21 is arranged so that R filter unit 21a, G filter unit 21b and B filter unit 21c may equally divide a circumferential line into three, where these filter units 21a, 21b and 21c transmit the light with wavelength band of R (red), G (green) or B (blue) respectively. The RGB filter unit 21 is composed such that by rotating the RGB filter unit 21 through the rotary motor 16, R filter unit 21a, G filter unit 21b, and B filter unit 21c are inserted continuously and almost sequentially into the light path, respectively.

As shown in FIG. 3A, R filter unit 21a, G filter unit 21b and B filter unit 21c have a filter characteristic each of which transmits the light of wavelength band of 600 to 700 nm, 500 to 600 nm, and 400 to 500 nm, respectively. In FIG. 3A, instead of reference symbols 21a, 21b, and 21c, reference symbols R, G, and B corresponding to the filter transmittance characteristics are used.

The fluorescence observation filter unit 22 is arranged on the circumferential direction so as to correspond to R1 filter unit 22a, G1 filter unit 22b and E1 filter unit 22c, where these filter units 22a, 22b and 22c transmit red light (R1) of narrow wavelength band, green light (G1) of narrow wavelength band or exciting light (E1) of narrow wavelength band, respectively. The fluorescence observation filter unit 22 is composed such that by rotating the filter unit 22 through the rotary motor 16, R1 filter unit 22a, G1 filter unit 22b, and E1 filter unit 22c are inserted continuously and almost sequentially into the light path, respectively.

As shown in FIG. 3B, R1 filter unit 22a, G1 filter unit 22b and E1 filter unit 22c have a filter characteristic each of which transmits the light of wavelength band of 590 to 610 nm, 540 to 560 nm, and 390 to 440 nm, respectively. In FIG. 3B, instead of reference symbols 22a, 22b, and 22c, reference symbols R1, G1, and E1 corresponding to the filter transmittance characteristics are used.

The illumination light from light source apparatus 3A is transmitted to a tip portion side of the insertion portion 7 of an electronic endoscope 2A by a light guide fiber 9 arranged in the electronic endoscope 2A. The light guide fiber 9 is formed with, for example, multi-component—glass fiber, a quartz fiber, etc. The light guide fiber 9 transmits the illumination light for normal observation and the illumination light for fluorescence observation with little transmission loss.

The light transmitted to the tip portion surface of the light guide fiber 9, is diffused and irradiated to a part for observation in the abdominal cavity through an illumination lens 24 attached on an illumination aperture which is faced to the surface at the tip portion.

In the tip end portion 8, an observation window is arranged adjacent to the illumination window. Behind the observation window of the tip end portion 8, an objective lens system 25 for forming an optical image, an aperture stop 26 which limits spatially an amount of incident light in order to perform focusing from a far distant point to a pericenter, an exciting light cutoff filter unit 27 which cuts off exciting light, and a charge-coupled device (CCD) 28 for performing, for example, a monochrome-image-pick-up (or white-black image-pick-up), as an image sensor which picks up each image of fluorescence and reflecting light are arranged.

As an image sensor which picks up the image of the fluorescence and the reflecting light, CMD (Charged Modulation Device) image sensor, C-MOS image sensor, AMI (Amplified MOS Imager), BCCD (Back Illuminated CCD), SPD (Single Photon Detector), etc. may be used instead of CCD 28.

The exciting light cutoff filter unit 27 is a filter unit which irradiates an observation object in order to excite fluorescence when a fluorescence observation is carried out, and shades the exciting light reflected by the observation object. Characteristic of the exciting light cutoff filter unit 27 is shown in FIG. 3C. The exciting light cutting filter unit 27 transmits the light of the wavelength band of 470 to 700 nm. That is, it has a characteristic which transmits visible light except some wavelength (390 to 470 nm) of blue ray band.

Furthermore, in the electronic endoscope 2A, a scope switch 29 which carries out instruction and operation for selecting a fluorescence image mode and a normal image mode, and carries out instruction and operation for freezing and releasing is arranged. A manipulating signal from the scope switch 29 is inputted into a controlling circuit 37 in an image processing apparatus 4A. The controlling circuit 37 is composed so that control action corresponding to the manipulating signal may be carried out.

For example, when a user operates a normal mode switch of the mode change switch in the scope switch 29, the controlling circuit 37 carries out the following control action. By control of the controlling circuit 37, the light source apparatus 3A becomes in a state, where the illumination light in the normal mode, that is light of R, G and B, is sequentially supplied to the light guide fiber 9.

FIG. 4A shows an intensity of light on the light receiving surface (image pick-up surface) of CCD 28 when an image of a white object 62 such as a white board as a standard object, is picked up in the normal mode. In this case, illumination of R, G, and B light is carried out by R filter unit 21a, G filter unit 21b and B filter unit 21c, each of which has a characteristic shown in FIG. 3A. Here, as shown in FIG. 3C, the filter characteristic of the exciting light cutoff filter unit 27 arranged ahead of CCD 28 has a characteristic such that all the light of G (green) and R (red) is transmitted, while as for the light of B (blue), only a part of light at a long wavelength side is transmitted. Therefore, the intensity of light on a light receiving surface (image pick-up surface) of CCD 28 becomes such that a short wavelength side of the light of B (blue) is cut off as shown by two point chain lines in FIG. 4A. That is, CCD 28 receives only the light of a part at the long wavelength side to the light of B (blue) as shown by a solid line. Therefore, also in the objective lens 25 which has an exciting light cutoff filter unit 27, it is composed so as to enable to carry out a normal observation.

Moreover, when a user operates the fluorescence mode switch of the mode change switch in the scope switch 29, the controlling circuit 37 carries out the following control action. By control of the controlling circuit 37, the light source apparatus 3A will be in the state where the illumination light of the fluorescence mode, i.e., the light of R1, G1, and E1 is sequentially supplied to the light guide fiber 9.

FIG. 4B shows an intensity of light on the light receiving surface (an image pick-up surface) of CCD 28 when an image of a skin is picked up in the fluorescence mode.

In this case, light having wavelength range of R1, G1, and E1 is illuminated by R1 filter unit 22a, G1 filter unit 22b and E1 filter unit 22c shown in FIG. 3B. Here, since the reflecting light by the light which passed through R1 filter unit 22a and G1 filter unit 22b is in the transmission zone of an exciting light cutoff filter unit 27, the light is received by CCD 28 according to the reflective characteristic of the skin. However, the reflecting light by the exciting light of E1 filter unit 22c is cut off since it is positioned outside of the transmission zone of an exciting light cutoff filter unit 27 as shown by two-point-chain-lines in FIG. 4B. As for the fluorescence emitted from the object for observation by the exciting light, the light in the transmission zone of the exciting light cutoff filter unit 27 is received by CCD28. As each reflecting light intensity of the illumination light by R1 filter unit 22a and G1 filter unit 22b is extremely small compared with the reflecting light intensity of the exciting light of E1 filter unit 22c, it is shown in magnification ratio of 100 (notation of ×100) in FIG. 4B. According to the present invention, the intensity of the light of the wavelength range of R1 and G1 by R1 filter unit 22a and G1 filter unit 22b is 1/100 of or less than that of the exciting light in the wavelength range of E1 by E1 filter unit 22c. Therefore, the intensity of the light in the wavelength ranges of R1 and G1 by R1 filter unit 22a and G1 filter unit 22b, and the intensity of fluorescence are shown in magnification ratio of 100 in FIG. 3B and FIG. 4B.

By this, the reflecting light intensity of the light and the fluorescence intensity which reach CCD 28 can be made near. Therefore, it is possible to avoid that only the CCD output of the reflecting light is saturated. However, since R1 filter unit 22a and G1 filter unit 22b are of low transmittance, an influence on variation on the light intensity by variation during manufacture becomes large.

The CCD 28 is driven with a CCD drive signal from the CCD drive circuit 31 arranged in the image-processing-apparatus 4A and outputs an image signal by conversing photo-electrically an optical image formed on the CCD 28.

A lost part of this image signal during cable transmission is amplified through the preamplifier 32 as a signal input means arranged in the image processing apparatus 4A. Moreover, the image signal is further amplified to a predetermined level through an automatic gain control (AGC) circuit 33. Then, an image signal is converted into a digital signal (image data) from an analog signal by an A/D conversion circuit 34. Each converted image data is temporarily stored (memorized) in a first frame memory 36a, a second frame memory 36b, and a third frame memory 36c through a multiplexer 35 which carries out switching.

The motor 16 for rotation is controlled by a controlling circuit 37, and outputs an encoding signal of an encoder attached to a revolving shaft of the motor 16 for rotation, etc., which is not illustrated, to the controlling circuit 37. The controlling circuit 37 controls a CCD drive circuit 31, switching of the multiplexer 35, etc. by synchronizing with the output of the encoder.

Moreover, the controlling circuit 37 controls switching of the multiplexer 35. In a normal mode, it controls so that each image signal picked up under illumination by R filter unit 21a, G filter unit 21b and B filter unit 21c, is sequentially memorized in the first frame memory 36a, the second frame memory 36b, and the third frame memory 36c respectively.

Also in a fluorescence mode, the controlling circuit 37 controls switching of the multiplexer 35. It controls so that each image signal picked up under illumination by R1 filter unit 22a, G1 filter unit 22b and E1 filter unit 22c, is sequentially memorized in the first frame memory 36a, the second frame memory 36b and the third frame memory 36c respectively.

The image signals stored in the frame memories 36a-36c are inputted into an image processing circuit 38. In the fluorescence image mode, the image processing circuit 38 carries out image processing for converting an input signal into an output signal having a hue which is easy to identify a normal tissue portion and a diseased tissue portion which is pathologically changed. Then, the image signal is converted into an analog RGB signal by the D/A conversion circuit 39, and is displayed on the monitor 5.

In this embodiment, as for the image processing apparatus 4A, it is composed such that three image signals, as a fluorescence image mode, that is, the image signals of the reflecting light image which are picked up from the reflecting light in the living tissue by two illumination light rays G1 and R1 of a narrow band range, and the image signal of the fluorescence image which picked up from the fluorescence generated from the living tissue by the exciting light E1 are inputted into the preamplifier 32 which is a signal input means.

In this embodiment, the image processing circuit 38 is composed such that a composite image is generated by allocating an image signal of the reflecting light (wavelength band containing a non-absorption band of the light of hemoglobin) by the illumination light by R1 filter unit 22a to B (blue) channel of RGB channel, an image signal of a fluorescence image to G (green) channel, and the image signal of the reflecting light (wavelength band containing an absorption zone of the light of hemoglobin) by the illumination light in G1 filter unit 22b to R (red) channel, and by composing them as one image as a composite means. Furthermore, in this embodiment, the image processing circuit 38 is composed so as to control a gain of three image signals inputted as mentioned later.

In the image processing apparatus 4A, the light adjusting circuit 40 which controls automatically the amount of opening of an aperture stop 13 for the light source in the light source apparatus 3A based on the signal through a preamplifier 32 is arranged. The light adjusting circuit 40 is controlled by the controlling circuit 37. Moreover, the controlling circuit 37 controls lamp current which drives an luminescence of the lamp 12 of the lamp drive circuit 11. Furthermore, the controlling circuit 37 is composed so that control action according to the operation of the scope switch 29 may be carried out.

Moreover, the electronic endoscope 2A has a scope ID generating section 23 which generates peculiar ID information which contains at least ID for the model itself. A model-type detection circuit 42 linked to the scope ID generating section 23 is arranged in the image processing apparatus 4A. The model-type detection circuit 42 is composed so as to detect the model information of connected electronic endoscope 2A and transmit the model information to a controlling circuit 37 when the electronic endoscope 2A is connected to the image processing apparatus 4A.

The controlling circuit 37 outputs a control signal for setting parameters, such as a matrix conversion of the image processing circuit 38, as a suitable one according to characteristics of the model of the electronic endoscope 2A connected. The setting switch 43 by which parameters, such as the matrix conversion, can be selected is connected to the image processing circuit 38.

As mentioned above, in the endoscope apparatus 1A, filter units which have been set so as to have the filter characteristics shown in FIG. 3A-FIG. 3C are used, as the normal observation filter unit 21 of the switching filter unit 17 of the light source apparatus 3A, the filter unit for fluorescence observation 22 and the exciting light cutoff filter unit 27 arranged at the imaging optical path of the electronic endoscope 2A. Thereby, a degree of distinction between portions of a normal tissue and a diseased tissue can be enlarged.

In FIG. 5A, an example of characteristic of an intensity distribution to the wavelength of the fluorescence image obtained by a living tissue is shown. In FIG. 5B, an example of characteristic of an intensity distribution to the wavelength of the reflecting light obtained by the living tissue is shown.

As seen from FIG. 5A, the intensity distribution characteristic of a fluorescence image has a peak near 520 nm. In this embodiment, the transmission characteristic by the exciting light cutoff filter unit 27 is set up so that the wavelength band near 520 nm may be included.

The intensity distribution characteristic of the reflecting light shown in FIG. 5B has a large absorption by hemoglobin near 550 nm, and forms a valley where a reflective intensity falls near such wavelength. A portion near 600 nm is considered as a non-absorption zone by hemoglobin. The center of wavelengths of two filter units 22a and 22b (G1, R1 in FIG. 5) is set as 550 nm and 600 nm. That is, in this embodiment, R1 filter unit 22a is set at a portion with the low absorbance of oxygenated hemoglobin in a transmitted wave length band, and G1 filter unit 22b is set at a portion with the high absorbance of oxygenated hemoglobin in the transmitted wave length band.

Furthermore, as for the light of G1 and R1 used as the reflecting light by the first and second normal illumination light which is illuminated in a fluorescence mode and are picked up by the reflecting light, the wavelength interval is set to 20 nm. It may be set to 20 nm or less. Moreover, the center of the wavelength of R1 filter unit 22a may be set to 610 nm.

A transmittance of the light of the blue zone (long wavelength band) which is shaded by the E1 filter unit 22c, and the transmittance of the light of the blue zone (short wavelength band) which is shaded by the exciting light cutoff filter unit 27 are set to 0.01% or less, respectively.

An image processing circuit 38 has a reflecting light color tone control circuit 54 as the first color control means, and the fluorescence color control circuit 58 as the second color control means. The reflecting light color tone circuit 54 has LUT (look-up table) 51, a parameter determination portion 52, and ROM 53. The reflecting light color tone control circuit 54 has LUT (look-up table) 55, a parameter determination portion 56, and ROM 57.

LUTs 51 and 55 are connected to ROMs 53 and 57 through the parameter determination portions 52 and 56. The parameter determination portions 52 and 56 are connected to a controlling circuit 37 and a setting switch 43. Two or more kinds of output values are stored beforehand in the ROMs 53 and 57, and values determined, through parameter determination portions 52 and 56, by the control signal of a controlling circuit 37 and by setup of the setting switch 43 is set in LUTs 51 and 55.

In this embodiment, a standard intensity ratio of the image signal of the reflecting light image of each wavelength band is stored in ROM 53. An intensity of the image signal of the reflecting light image of each wavelength band can be adjusted so that an intensity ratio of the image signal of the reflecting light image of each wavelength band obtained when a standard object is used as an object becomes the standard intensity ratio. Moreover, the standard intensity ratio of the image signal of a reflecting light image and the image signal of a fluorescence image by which the color control is carried out by the reflecting color control circuit 54 is stored in ROM 57. An intensity of the image signal of the fluorescence image obtained when a living tissue is used as an object, can be adjusted so that it may become a standard intensity ratio of the image signal of a reflecting light image and the image signal of a fluorescence image by which the color control is carried out by the reflecting color control circuit 54.

In case of the fluorescence mode, output values corresponding to three signals which are inputted from input terminals Ta-Tc are read out by LUTs 51 and 55, and they are outputted to R, G, and B channels from output terminals Ta″, Tb″, and Tc.″. In case of the normal mode, look-up tables 51 and 55 are set to ones having characteristics which output an input signal as it is.

An image data outputted to R, G, and B channels from output terminals Ta″, Tb″ and Tc″ is converted into an analog RGB signal by the D/A conversion circuit 39, and is displayed on the monitor 5, and it is displayed as a composite image by this monitor 5.

The setting switch 43 has the first color control switch 59 and the second color control switch 60, and it is composed such that either of the switches can be selected. The first color control switch 59 is connected with the reflecting color control circuit 54. The second color control switch 60 is connected with the fluorescence color control circuit 58. When the first color control switch 59 is selected, the color control processing of the reflecting light by the reflecting color control circuit 54 is carried out, and when the second color control switch 60 is selected, the color control processing of the fluorescence by the reflecting color control circuit 60 is carried out.

Here, a concrete color control processing using the endoscope apparatus of this embodiment is explained.

First, a color control of the reflecting light is carried out.

A user arranges a standard object 62 of the tip portion of an electronic endoscope A2. Then, the first color control switch 59 is selected. In the reflecting light color tone control circuit 54, the following color controls (determination of a coefficient alpha) are carried out to the R1 reflecting light signal (Ta), the G1 reflecting light signal (Tb), and fluorescence (Tc) of the standard object by an exciting light E1 obtained when a standard object is used as an object. Here, alpha is a coefficient used as Ta′=Tb′.
Ta′=Ta×α
Tb′=Tb
Tc′=Tc

In the fluorescence color control circuit 58, a color control is not carried out, but an output signal is converted.
Ta″=Tb′
Tb″=Tc′
Tc″=Ta′

Thereby, a color control is carried out so that the intensity ratio of the R1 reflecting light signal Ta and the G1 reflecting light signal Tb may become a predetermined intensity ratio.

Then, a color control of fluorescence is carried out.

The user arranges a living tissue at the object 62 of the tip portion of an electronic endoscope A2. Subsequently, an area of interest 61 of the normal tissue of the living tissue is set up, and the second color control switch 60 is selected.

In the fluorescence color tone control circuit 58, the following color controls (determination of a coefficient β) are carried out to the R1 reflecting light signal (Ta′), the G1 reflecting light signal (Tb′) which are average value signals of the area of interest 51, and fluorescence (Tc′) of the standard object by exciting light E1.
Ta″=Tb′=Tb
Tb″=Tc′×β=Tc×β
Tc″=Ta′=Ta×α

Thereby, a color control is carried out so that the intensity ratio of G1 reflecting light signal Tb and the intensity ratio of a fluorescence signal to the reflecting light signal adjusted to the predetermined intensity ratio may become a predetermined intensity ratio.

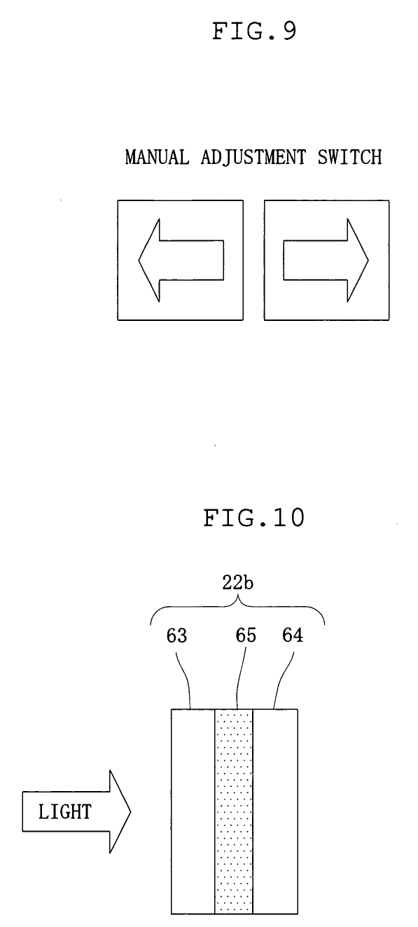

Color control (α, β) is determined by two steps of adjustment mentioned above. The image processing circuit 38 carries out the color control of an image signal by using the value of α and β, and the fluorescence image after performing the color control is displayed on the monitor 5. Thereby, a user can carries out the observation in the fluorescence mode. As for the determination of the value of β, it may be composed such that the color control switch 60 may increase or decrease the value of β according to the direction of an arrow mark as shown in FIG. 9 so that the user can set up manually according to the user's liking.

The image data which is outputted to R, G, B channels is converted into analog RGB signal by the D/A conversion circuit 39 and it is outputted to the monitor 5, and then it is indicated by a spurious color as a composite image by this monitor 5.

As a result, in the image processing apparatus 4A of this embodiment, a composite image which is easy to identify a normal tissue and a diseased tissue can be obtained by adjusting the gain of three image signals in the image processing circuit 38, That is, according to the fluorescence endoscope apparatus of this embodiment, it is adjusted by the image processing circuit 38 when the intensity ratio of only the image signal of the reflecting light image of each wavelength band chooses the first color control switch 59 as a state using a changeless standard object.

Since the whole state of a standard object is being fixed uniformly, it is not necessary to set up a zone into an observation image, and a color control (for raising a color reproduction) of the reflecting light can be simply and exactly carried out.

Moreover, the intensity of the image signal of the fluorescence image can be adjusted by the image processing circuit 38 when the second color control switch 60 is selected so that an intensity ratio of the image signal of the fluorescence image obtained by radiating the exciting light for exciting fluorescence in a patient's body (living tissue), and the image signal of the reflecting light image of a plurality of wavelength bands, where the intensity ratio has been adjusted by the image processing circuit 38 when the first color control switch 59 is selected by using the standard object, may become a predetermined intensity ratio. Therefore, a constant and exact color reproduction can be carried out and a useful diagnostic information can be obtained, even if a fluorescence intensity differs for every patient. If only the intensity of the image signal of the fluorescence image obtained from the living tissue is adjusted, it is not necessary to set up the zone of a pathological change portion with a difficult setup, it will be sufficient for a setup to perform the setup of range of only the normal portion of an easy living tissue, and a color control will become possible from the value of the image signal of the fluorescence image in the normal portion.

As mentioned above, correction (color control) of the color tone variation by variation generated during manufacture of R1 filter unit 22a, G1 filter unit 22b etc., and different fluorescence intensity variation for every patient can be carried out simply and exactly. Therefore, according to the image processing apparatus 4A of the present invention, an image by which a normal tissue or a diseased tissue is easily identified with simple constitution can be achieved.

Here, the image processing circuit 38 can be composed so as to composite an image as one, wherein an image signal at the short wavelength side of a reflecting light (wavelength band containing the absorption zone of the light of hemoglobin) is assigned to B channel of RGB channel, an image signal of a fluorescence image is assigned to G channel, and an image signal by the long wavelength side of a reflecting light (wavelength band containing the non-absorption zone of the light of hemoglobin) is assigned to R channel. In the present embodiment, as for the image processing apparatus 4A, the present invention is applied to what is composed using look-up tables 51 and 55 in the image processing circuit 38. However, the present invention is not limited to this. The present invention may be applied to what is composed using a matrix circuit or color tone conversion in the image processing circuit 38.

Furthermore, in this embodiment, the image processing apparatus 4A is composed so as to adjust a gain of three image signals inputted by the image processing circuit 38. However, the present invention is not limited to this. It may be composed, for example, so that the gain of three image signals inputted may be adjusted in a preamplifier 32, an auto gain control (AGC) circuit 33, or D/A conversion circuit 39, etc.

Moreover, in the normal observation mode, a reflecting color control circuit 54 for color control can be used.

A user arranges a standard object 62 of the tip portion of an electronic endoscope A2. Then, the first color control switch 59 is selected. In a reflecting light color tone control circuit 54, the following color controls (determination of coefficient α′, β′) are carried out to R reflecting light signal (Ta), G reflecting light signal (Tb), and B reflecting light signal (Tc) which are obtained when the standardobject is used as an object.
Ta′=Ta×α′
Tb′=Tb
Tc′=Tc×β′

The fluorescence color control circuit 58 outputs an input signal without converting it.
Ta″=Ta′
Tb″=Tb′
Tc″=Tc′

By such way as mentioned above, the color tone variation caused by variation in manufacture of the normal observation filter unit 21 and the like can be corrected without adding any circuit.

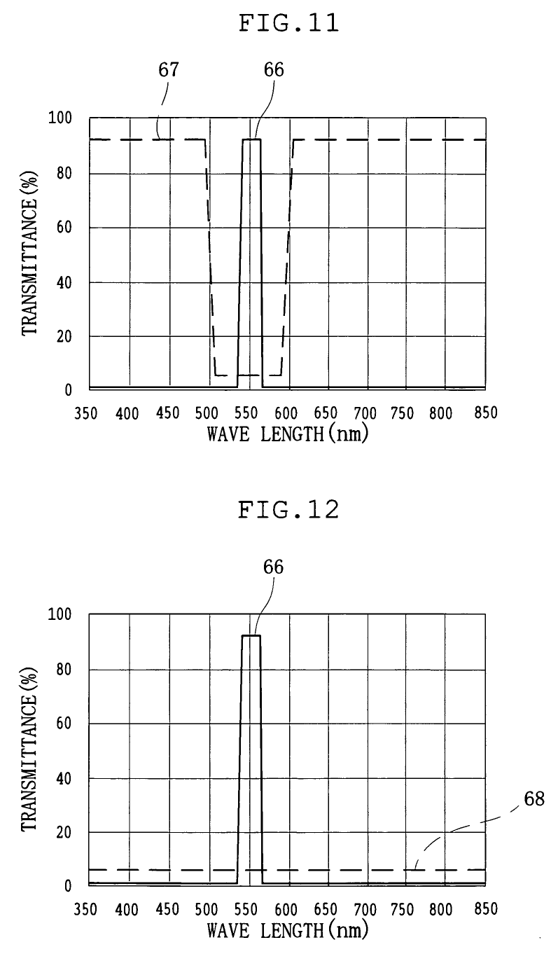

FIG. 10 is an outline composition diagram of G1 filter unit used for the fluorescence endoscope apparatus concerning the second embodiment of the present invention. FIG. 11 is a graph showing a transmittance characteristic of an optical filter unit shown in FIG. 10. FIG. 12 is a graph showing a transmittance characteristic of a modification of an optical filter unit shown in FIG. 11. As shown in FIG. 10, .G1 filter unit 22b of this embodiment is composed such that an optical filter unit 63 and an optical filter unit 64 are joined through adhesives 65.

The multilayered film coat of dielectrics (SiO₂, Ta₂O₅ etc.) is given to the optical filter unit 63. As shown in a reference numeral 66 of FIG. 11, it is composed of a band pass filter unit which transmits only the light of wavelength of 540 nm to 560 nm. The multilayered film coat of dielectrics (SiO₂, Ta₂O₅ etc.) is given to the optical filter unit 64. As shown in a reference numeral 67 of FIG. 11, it is composed of a band cutoff filter unit in which transmittance of the light of wavelength of 540 nm to 560 nm becomes 0.8%.

Manufacture using coat deposition apparatus can be realized by using the multilayered film coat of dielectrics. It is possible to reduce the variation during manufacture which is 0.8% of transmittances compared with the ND filter unit which absorbs light.

It is possible to use a coat of a metal monolayer (nickel etc.) which has a characteristic as shown in a reference numeral 68 of FIG. 12 as a modification of the optical filter unit 64. As for the R1 filter unit 22a, it can be composed such that two sheets of an optical filter unit are joined through adhesives like the G1 filter unit 22b.

In this embodiment, the transmittances of the R1 filter unit 22a and the G1 filter unit 22b are set to about 0.8%. That is, it is set to 1/100 or less of the transmittance of the filter unit for exciting light (the E1 filter unit 22c). By this way, an intensity of the reflecting light by the R1 filter unit, an intensity of the reflecting light by the G1 filter unit and an intensity of the fluorescence by the E1 filter unit, wherein each of the light reaches the CCD 28, can become nearly same. Therefore, it is possible to avoid that only the CCD output of the reflecting light is saturated.

In this embodiment, each of the R1 filter unit 22a and the G1 filter unit 22b is composed by joining two sheets of an optical filter unit. Thereby, design and manufacture of a multilayered film coat using both surfaces, that is totally four surfaces, of each optical filter unit can be realized, and the design and the manufacture of the coat become easy. Moreover, it is also possible to manufacture highly precise R1 filter unit 22a and G1 filter unit 22b by joining an optical filter unit in combination which offsets manufacture errors based on a measurement of the manufacture error of each optical filter unit.

By this way mentioned above, the R1 filter unit 22a and the G1 filter unit 22b which have low transmittance can be manufactured with high precision. Thereby, an electric noise generated in the color control of the image processing apparatus 38 can be reduced.

1. A fluorescence endoscope apparatus comprising,
a light source apparatus having at least a light source, an optical filter unit for exciting light, and a plurality of optical filter units for normal illumination light,
an electronic endoscope which leads exciting light and a plurality of normal illumination light from the light source apparatus to an object, and picks up an image of fluorescence and a plurality of reflecting light obtained from the object,
and an image processing apparatus which processes an image signal of a fluorescence image and a plurality of reflecting light images picked up by the electronic endoscope, and delivers them to a monitor, and
the image processing further comprising a first color control means which carries out a color control of only the image signals of a plurality of the reflecting light images based on an image signal obtained by using a standard object as an object.

2. The fluorescence endoscope apparatus according to claim 1, wherein the image processing apparatus comprises a second color control means which carries out a color control of an image signal of said plurality of reflecting light images adjusted by the first color control means and an image signal of the fluorescence image obtained by using a living tissue as an object on the basis of the image signal obtained by said image processing apparatus using the living tissue as the object.

3. The fluorescence endoscope apparatus according to claim 1, wherein transmittances of the plurality of optical filter unit for normal illumination light is 1/100 or less of the transmittance of the optical filter unit for the exciting light.

4. The fluorescence endoscope apparatus according to claim 2, wherein transmittances of the plurality of optical filter unit for normal illumination light is 1/100 or less of the transmittance of the optical filter unit for the exciting light.

5. The fluorescence endoscope apparatus according to claim 1, wherein each of optical filter units for the normal illumination light is composed of two sheets of the optical filter unit which are pasted together

6. The fluorescence endoscope apparatus according to claim 2, wherein each of optical filter units for the normal illumination light is composed of two sheets of optical filter unit which are pasted together.

7. The fluorescence endoscope apparatus according to claim 3, wherein each of optical filter units for the normal illumination light is composed of two sheets of optical filter unit which are pasted together.

8. The fluorescence endoscope apparatus according to claim 4, wherein each of optical filter units for the normal illumination light is composed of two sheets of the optical filter unit which are pasted together.
